(19) United States
(12) Patent Application Publication
(10) Pub. No.: US 2010/0106215 A1
(43) Pub. Date: Apr. 29, 2010

(54) SYSTEMS AND METHODS TO DETECT IMPLANTABLE MEDICAL DEVICE CONFIGUARATION CHANGES AFFECTING MRI CONDITIONAL SAFETY

(76) Inventors: Scott R. Stubbs, Maple Grove, MN (US); Diane Schuster, Bloomington, MN (US); Jean M. Bobgan, Maple Grove, MN (US); Ronald D. Berger, Baltimore, MD (US)

Correspondence Address: FAEGRE & BENSON LLP;PATENT DOCKETING - INTELLECTUAL PROPERTY (32469), 2200 WELLS FARGO CENTER, 90 SOUTH SEVENTH STREET, MINNEAPOLIS, MN, 55402-3901, US

(21) Appl. No.: 12/559,132

(22) Filed: Sep. 14, 2009

Related U.S. Application Data
(60) Provisional application No. 61/107,908, filed on Oct. 23, 2008.

(51) Int. Cl. A61N 1/37

(52) U.S. Cl. 607/37

(57) ABSTRACT

Systems and methods for checking the connection of a lead to an implantable medical device implanted within a patient's body are disclosed. An illustrative method includes measuring at least one characteristic associated with the lead connection to the implantable medical device prior to an MRI scan. The method further includes comparing the at least one measured characteristic with a threshold parameter programmed within the implantable medical device. The method further includes setting a flag in the implantable medical device upon the at least one measured characteristic satisfying at least one condition associated with the threshold parameter for a predetermined period of time. The flag indicates a disconnection of the lead from the implantable medical device.

### CROSS-REFERENCE TO RELATED APPLICATION

This application claims priority to Provisional Application No. 61/107,908, filed Oct. 23, 2008, which is herein incorporated by reference in its entirety.

### TECHNICAL FIELD

The present invention pertains to implantable medical devices. More particularly, the present invention relates to systems and methods for detecting configuration changes affecting MRI conditional safety in implantable medical devices.

### BACKGROUND

Magnetic resonance imaging (MRI) is a non-invasive imaging method that utilizes nuclear magnetic resonance techniques to render images within a patient's body. Typically, MRI systems employ the use of a magnetic coil having a magnetic field strength of between about 0.2 to 3.0 Tesla. During the procedure, the body tissue is briefly exposed to radio frequency (RF) pulses of electromagnetic energy in a plane perpendicular to the magnetic field. The resultant electromagnetic energy from these pulses can be used to image the body tissue by measuring the relaxation properties of the excited atomic nuclei in the tissue.

The physical configuration of an active implantable medical device (AIMD) constitutes one element of a safe environment for MRI scans. In some systems, the AIMD may include a number of lead wires that connect to human tissue for providing stimulus therapy to the patient, and/or for sensing various parameters within the patient's body. In certain systems, for example, the AIMD may include a number of leads that deliver electrical stimulus energy for pacing a patient's heart and/or for delivering electrical shocks to the heart in response to an adverse event. These lead wires are often part of the physical system approved for an MRI scan.

Under some circumstances, the lead wires may need to be replaced independently from the remainder of the system. In some cases, safety risks such as tissue heating from the electrode tip may arise if the implanting physician does not replace the existing lead wire with an MRI approved lead wire. Furthermore, safety risks such as excessive vibration and torque movements may arise if lead wires are abandoned within the body during replacement. Additionally, safety risks may arise if an MRI authorization process is skipped after a lead wire is replaced. In some systems, the impedance of the lead wires is verified at the time of an MRI scan to determine if the lead impedance is within an acceptable range. However, verifying the lead impedance at the time of an MRI scan does not indicate whether a change in the lead configuration had been made prior to the MRI scan, which can render the implantable device MRI conditionally unsafe.

### SUMMARY

The present invention relates generally to systems and methods for detecting configuration changes affecting MRI conditional safety in implantable medical devices. Embodiments of the present invention include systems and methods for checking the connection of a lead to an implantable medical device implanted within a patient's body. An illustrative method includes measuring at least one characteristic associated with the lead connection to the implantable medical device prior to an MRI scan. The method further includes comparing the at least one measured characteristic with a threshold parameter programmed within the implantable medical device. The method further includes setting a flag in the implantable medical device upon the at least one measured characteristic satisfying at least one condition associated with the threshold parameter for a predetermined period of time. The flag may be used to indicate a disconnection of the lead from the implantable medical device prior to the patient undergoing an MRI scan.

### BRIEF DESCRIPTION OF THE DRAWINGS

FIG. 1 shows an example system including an implantable medical device and remote terminal that can be used in relation to embodiments of the present invention;

FIG. 2 is an example MRI process in which a new medical device is implanted into a patient;

FIG. 3 is a diagram showing an example lead revision scenario;

FIG. 4 is a diagram showing an example implant device revision;

FIG. 5 shows an example system that can be used in relation to embodiments of the present invention;

FIG. 6 is a schematic diagram of an example computing device upon which embodiments of the present invention may be implemented; and

FIG. 7 shows an example method for detecting the disconnect of a lead from a pulse generator (PG).

### DETAILED DESCRIPTION

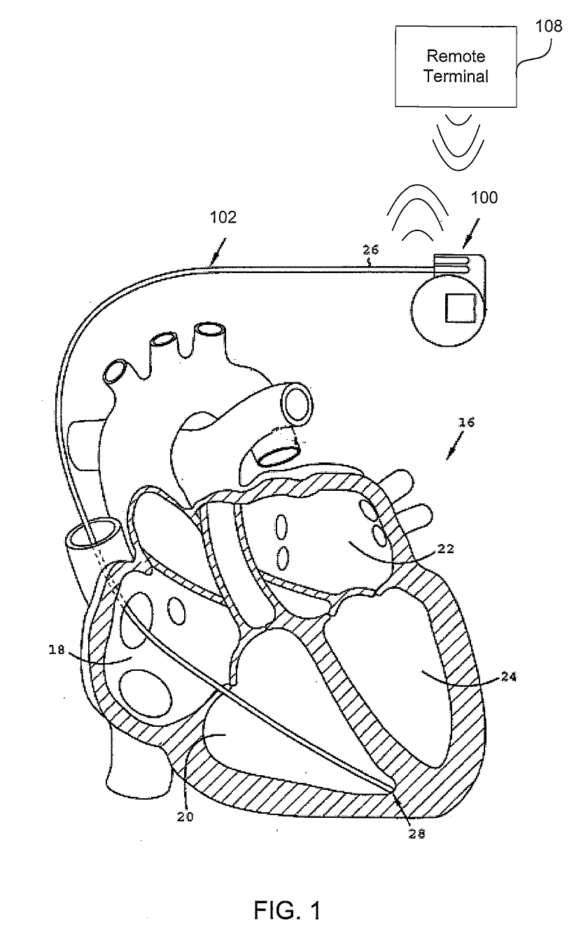

FIG. 1 is a schematic view of an illustrative medical device 100 equipped with a lead implanted within the body of a patient. In the illustrative embodiment depicted, the medical device 100 is a PG implanted within the body. The PG includes a lead 102 placed in the patient's heart 16. The heart 16 includes a right atrium 18, a right ventricle 20, a left atrium 22, and a left ventricle 24. The PG 100 can be implanted subcutaneously or submuscularly within the body, typically at a location such as in the patient's chest or abdomen, although other implantation locations are possible.

A proximal portion 26 of the lead 102 can be coupled to or formed integrally with the PG 100. A distal portion 28 of the lead 102, in turn, can be implanted within a desired location within the heart 16 such as the right ventricle 20, as shown. Although the illustrative embodiment depicts only a single lead 102 inserted into the patient's heart 16, in other embodiments multiple leads can be utilized so as to electrically stimulate other areas of the heart 16. In some embodiments, for example, the distal portion of a second lead may be implanted in the right atrium 18. In addition, or in lieu, another lead may be implanted at the left side of the heart 16 (e.g., in the coronary veins) to stimulate the left side of the heart 16. Other types of leads such as epicardial leads may also be utilized in addition to, or in lieu of, the lead 102 depicted in FIG. 1.

During operation, the lead 102 can be configured to convey electrical signals between the heart 16 and the PG 100. For example, in those embodiments where the PG 100 is a pacemaker, the lead 102 can be utilized to deliver electrical therapeutic stimulus for pacing the heart 16. In those embodiments where the PG 100 is an implantable cardiac defibrillator, the lead 102 can be utilized to deliver electric shocks to the heart 16 in response to an event such as a heart attack. In some embodiments, the PG 100 includes both pacing and defibrillation capabilities.

The PG 100 is communicable wirelessly with one or more remote terminals 108 (e.g., a computing device and/or programming device) located outside of the patient's body. In embodiments, the PG 100 communicates with the remote terminal 108 via any suitable wireless communication interface. In certain embodiments, for example, the PG 100 is configured to communicate with the one or more remote terminals 108 via an RF, inductive, and/or an acoustic telemetry link.

Generally, MRI scanning of patients with implanted medical devices, such as the PG 100 in FIG. 1, is prohibited unless the implanted medical device includes a labeling system indicating that the implanted medical device is MRI conditionally safe. In some embodiments, for example, a labeling system includes specific physical configurations that must be met for an implanted medical device to be considered MRI conditionally safe. As an example, the labeling system may specify what type(s) of lead wires may be used for the PG 100, and that no lead wires are abandoned (e.g., lead wires are not disconnected from the PG 100 or any human tissue). Accordingly, the PG 100 is considered safe for scanning if the lead wire 102 connected to the PG 100 is the type of lead wire specified by the labeling system as MRI conditionally safe, and there are no abandoned leads present within the body.

In some embodiments, the PG 100 is configured to store patient data and lead configuration information that can be used to determine whether the configuration of the PG 100 is MRI conditionally safe. As an example, the patient data stored within the PG 100 can indicate when a PG 100 and/or lead configuration change has been made. In some embodiments, the patient data includes an MRI authorization flag. In some embodiments, when this MRI authorization flag is set to an approved state (e.g., 1), the configuration of the PG 100 and lead wire 102 is considered to be MRI conditionally safe. If the MRI authorization flag is set to an unapproved state (e.g., 0), then no MRI scan may be performed on the PG 100 until an examination of the PG 100 and lead wire 102 is performed by a clinician to determine if these components are MRI conditionally safe.

In some embodiments, the remote terminal 108 alerts the clinician of the status of the MRI authorization flag. As an example, the PG 100 transmits the status of the MRI authorization flag to the remote terminal 108, where the status of the MRI authorization flag is displayed on a user interface on the remote terminal. In alternative embodiments, the remote terminal 108 sounds an alarm upon receiving information from the PG 100 that the MRI authorization flag is in an unapproved state.

During the course of treatment, the PG or lead may be replaced (e.g., due to a fractured or broken lead). Lead revisions are common procedures and many physicians mix and match PGs and leads. If a lead is replaced, the new lead may not be approved in the system labeling for MRI scans. Additionally, a lead revision may include the abandonment of a lead within the body.

Generally, after a PG or lead revision, an MRI authorization process is performed to determine if the PG and lead are MRI conditionally safe after the revision. In some embodiments, the MRI authorization process includes updating the patient data within the PG and/or the remote terminal to indicate that a configuration change has been made, and set the MRI authorization flag if the PG and lead are determined to be MRI conditionally safe after the revision. The authorization flag can be set, for example, by a clinician performing the MRI authorization process. However, if this MRI authorization process is skipped, the MRI authorization flag may be left in an incorrect state indicating that the PG and leads are MRI conditionally safe (e.g., a proper combination of PG and leads) when both the PG and leads are actually MRI conditionally unsafe.

Accordingly, embodiments of the present invention detect when a lead is disconnected from the PG. In some embodiments, upon detection of the lead disconnect from the PG, the MRI authorization flag is set to an unapproved state to indicate that the PG and/or the lead wires may be MRI conditionally unsafe. In some embodiments, the MRI authorization flag can be set either manually by a clinician, or automatically by the PG itself.

In some embodiments, a lead disconnect from a PG is determined by checking the lead impedance of a lead wire connected to a PG. As an example, a lead disconnect can be detected when the lead impedance exceeds 2,000 ohms for a predetermined period of time. For example, a lead disconnect can be detected when the lead impedance exceeds 2,000 ohms for a period of greater than five seconds, ten seconds, or any other predetermined time period. In some embodiments, upon detection of a lead disconnect, the MRI authorization flag is set to an unapproved state in the patient data stored in the PG. In some embodiments, a lead disconnect can be detected when other lead impedance thresholds have been exceeded or when other desired time intervals have lapsed.

The lead impedance threshold may be exceeded for a specified period of time when a lead revision is taking place, or when there is a fractured lead. As discussed above, after a lead revision has occurred, the replaced lead may be considered an unapproved lead, and therefore MRI conditionally unsafe. Further, fractured leads may also present a hazard to patients when subjected to MRI scans. Thus, setting the MRI authorization flag to an unauthorized state upon detecting a lead disconnect prevents the treating physician or any other medical technician from performing an MRI on a PG that may be MRI conditionally unsafe.

In some embodiments, a lead disconnect is detected by utilizing a force sensor to measure the amount of force between a lead and a PG. Embodiments of the present invention use any desired force sensor such as the Honeywell FSS series of low profile force sensors. As an example, a lead disconnect is determined when a measured force between the lead and the PG is below a threshold for a specified period of time (e.g., five seconds). Other force thresholds and specified time periods may be used to indicate that a lead is disconnected from a PG.

An example of utilizing a force sensor to measure the force between a lead and a PG is disclosed in U.S. Pat. No. 7,047,075, entitled “Apparatus for Actively Monitoring Device for Lead Fixation in Implantable Tissue Stimulators,” the entire contents of which are incorporated herein by reference. In embodiments, the MRI authorization flag is set to an unapproved state upon the determination that the measured force between the lead and the PG has fallen below the force threshold for a specified period of time. For example, the MRI authorization flag may be set in the PG to indicate a lead disconnect, upon determining that the measured force between the lead and the PG has fallen below a force of between about 35 to 46 Newtons for a time period greater than 5 seconds. As discussed above, setting the MRI authorization flag to an unapproved state upon detecting a lead disconnect warns the treating physician or any other medical technician that the PG configuration may be MRI conditionally unsafe.

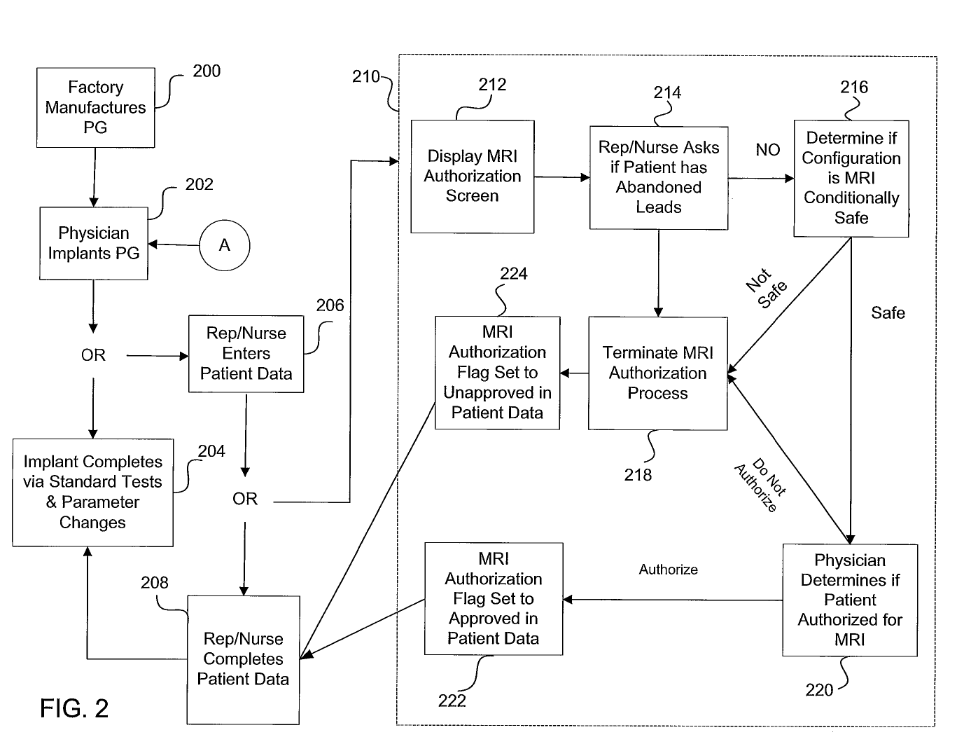

FIGS. 2-4 are diagrammatic views showing several example scenarios of lead and PG revisions. FIGS. 2-4 may illustrate, for example, when a lead disconnect occurs, and an example MRI authorization process that can be employed to determine whether the use of a lead and/or PG during an MRI scan is unsafe.

FIG. 2 illustrates an example process in which a new medical device (e.g., a lead) is implanted into a patient. The method starts when a factory manufactures a PG (block 200) and sets the MRI authorization flag in the PG as unapproved (e.g., 0). Hospitals may order the PGs from the factory where a cardiac physician implants (block 202) the PG into patients who need a particular treatment provided by the PG. After the physician implants the PG into a patient, the procedure is completed via standard tests to determine the integrity of the medical device (e.g., lead impedance, pace threshold, P/R wave intrinsic amplitude) and parameter changes to ensure proper operation of the device (e.g., pacing mode, pacing rate, atrial-ventricular delay) (block 204). Alternatively, a representative or nurse enters patient data (block 206) including performing an MRI authorization process (block 210). As an alternative to performing an MRI authorization process, the representative/nurse completes the patient data (e.g., patient name, information on the PG and leads, date of implant, etc.) (block 208).

FIG. 2 further illustrates an example MRI authorization process (block 210) that can be performed. The MRI authorization process (block 210) is initiated by displaying an MRI authorization screen (block 212). As an example, the MRI authorization screen appears in a user interface on the remote terminal 108 of FIG. 1. The MRI authorization screen asks if the patient has abandoned leads (block 214). If the representative/nurse indicates that there are abandoned leads, the MRI authorization process is terminated (block 218). If the representative/nurse indicates that there are no abandoned leads, the MRI authorization process determines if the PG configuration is MRI conditionally safe (block 216). If the MRI authorization process determines that the PG configuration is not MRI conditionally safe, the MRI authorization process is terminated (block 218). However, if the MRI authorization process determines that the PG configuration is MRI conditionally safe, then a physician determines if the patient is authorized for an MRI scan (block 220). If the physician determines that the patient is not authorized for an MRI scan, the MRI authorization process is terminated (block 218). However, if the physician determines that the patient is authorized for an MRI, then the MRI authorization flag in the patient data is set to an approved state (e.g., 1) (block 222). If the MRI authorization process is terminated, then the MRI authorization flag in the patient data is set to an unapproved state (e.g., 0) (block 224). Upon completion of the MRI authorization process, the representative/nurse completes the patient data (block 208). After the representative/nurse completes the patient data, the implant procedure is completed (block 204).

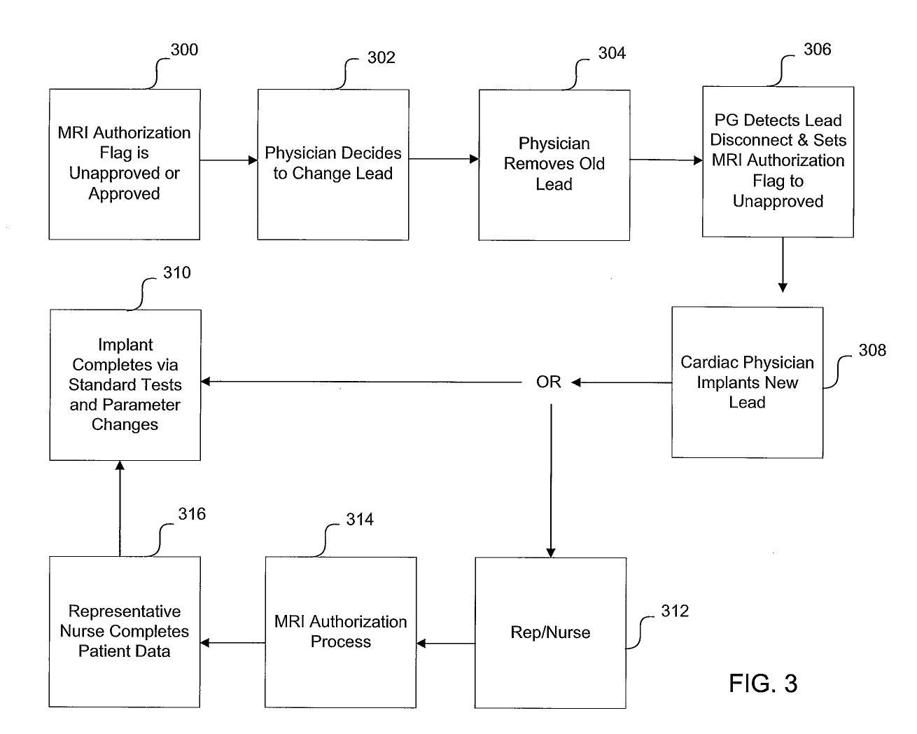

FIG. 3 is a diagram showing an example scenario of a lead revision. A lead revision can occur, for example, when leads connected to a PG need to be replaced, due to a failure of the lead. At the time of the lead revision, the MRI authorization flag may be in an unapproved or an approved state (block 300). The lead revision occurs when the treating physician determines that a lead connected to a pulse generator needs to be changed (block 302). The physician removes the old lead (block 304), which in some embodiments is detected by the PG as a lead disconnect, and then sets the MRI authorization flag to an unapproved state (block 306). The physician then implants the new lead (block 308). After the physician implants the new lead, the implant procedure is completed by a standard test and parameter changes similar to those discussed above (block 310). Alternatively, or in addition, a representative/nurse enters patient data (block 312), and performs an MRI authorization process (block 314). The MRI authorization process (block 314) may be conducted, for example, in a similar manner as described for the MRI authorization process discussed with respect to FIG. 2. After the MRI authorization process (block 314) is completed, the representative/nurse completes the patient data (block 316), and the implant procedure is subsequently completed (block 310).

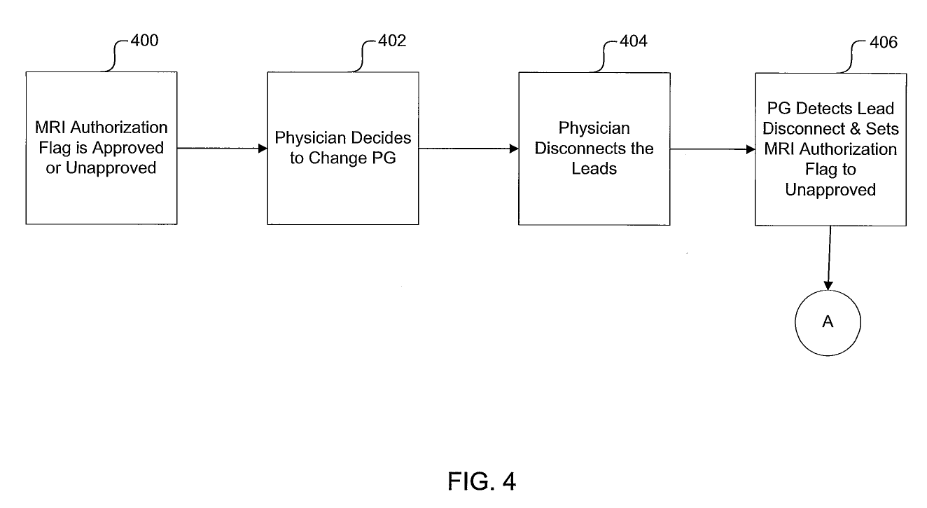

FIG. 4 is a diagram showing an example scenario for a PG revision. A PG revision can occur, for example, when the treating physician determines that the implanted PG needs to be replaced. At the time of the PG revision, the MRI authorization flag in the patient data may be set to an unapproved or approved state (block 400). The PG revision starts when the treating physician decides that the PG needs to be changed (block 402). During the PG revision, the physician disconnects the leads (block 404). When the leads are disconnected, the PG detects the lead disconnect and sets the MRI authorization flag to an unapproved state (block 406). After the PG sets the MRI authorization flag to an unapproved state, the implant procedure illustrated in FIG. 2 at point A (block 202) is repeated.

As illustrated in FIGS. 3 and 4, the ability to detect the lead disconnect permits the MRI authorization flag to be set to an unapproved state. If the treating physician relied on the representative/nurse to perform the MRI authorization process to set the MRI authorization flag to the appropriate state, and the MRI authorization process is skipped, then the MRI authorization flag may be left in an approved state even though the lead wires or the PG may be MRI conditionally unsafe. Further, when there is an unauthorized revision of the lead wires or PG by a party that does not perform the MRI authorization process, the ability to detect the lead disconnect permits the MRI authorization flag to be set to an unapproved state to indicate that the lead wires or PG may be MRI conditionally unsafe. Accordingly, by setting the MRI authorization flag to an unapproved state upon detection of a lead disconnect or lead failure, physicians or any other medical technician would be warned to check the PG and lead wires prior to conducting an MRI scan on the patient.

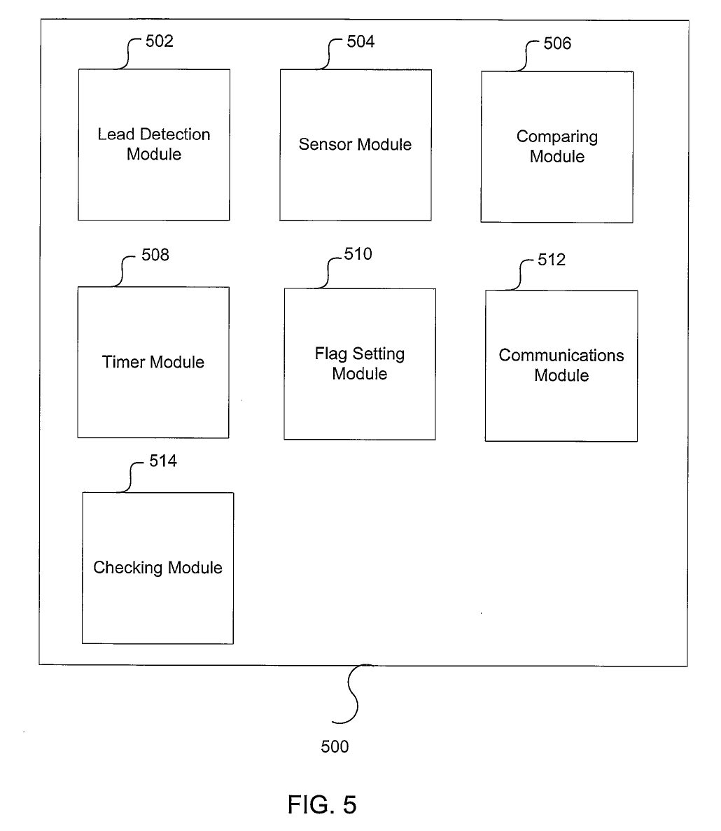

FIG. 5 illustrates an example system including modules that can be used with embodiments of the present invention. The term “module” refers broadly to a software, hardware, or firmware component (or any combination thereof). Modules are typically functional components that can generate useful data or other output using specified input(s). A module may or may not be self-contained. An application program (also called a “start application”) may include one or more modules and/or a module can include one or more application programs.

In some embodiments, the system 500 is incorporated in the PG 100 of FIG. 1. In alternative embodiments, the system 500 is incorporated in the remote terminal 108 of FIG. 1. In embodiments, the system 500 includes at least a lead detection module 502, a sensor module 504, a comparing module 506, a timer module 508, a flag setting module 510, a communications module 512, and a lead checking module 514. In some embodiments, the PG 100 of FIG. 1 includes one or more of the modules illustrated in system 500 of FIG. 5, while the remote terminal 108 of FIG. 1 includes one or more of the modules illustrated in system 500 of FIG. 5.

In certain embodiments, the lead detection module 502 performs one or more measurements to determine if a lead is properly connected to a PG. As an example, each measurement result is verified against a range of valid values until an in-range measurement has been detected. When an in-range measurement has been detected, the lead detection module 502 determines whether a lead has been attached to the PG. In some embodiments, the lead detection module 502 is initiated upon powering up the PG. As an example, when a PG is manufactured and shipped to a hospital, no leads may be attached to the PG. Thus, the lead detection module 502 is initiated upon powering up the PG to determine when leads are attached to the PG. In embodiments, when the PG is restarted, if the lead detection module 502 did not previously detect that a lead had been attached to the PG, then the lead detection module 502 is initiated upon restart of the PG.

In some embodiments, the lead detection module 502 performs lead impedance measurements to determine when a lead has been attached to the PG. As an example, a lead is detected when the lead detection module 502 measures a lead impedance between about 200 ohms to 2,000 ohms. In other embodiments, the lead detection module 502 performs force measurements to determine when a lead has been attached to the PG. As an example, a lead is detected when the lead detection module 502 measures a force between about 155 to 245 Newtons on the terminal pins inserted into the PG header.

After the lead detection module performs a measurement and does not detect a lead, the lead detection module 502 can be configured to perform the measurement again after a specified period of time (e.g. 2 seconds) to verify that the lead is not connected to the PG. In embodiments, after the lead detection module 502 determines that a lead has been attached to the PG, a lead detection flag is set (e.g., 1). In embodiments, when the lead detection flag is set, the system 500 performs a process (discussed below) to determine if the lead, which has been connected to the PG, is disconnected from the PG.

In embodiments, the sensor module 504 measures at least one characteristic associated with a lead connection to the PG. With respect to the illustrative system of FIG. 1, for example, the sensor module 504 may utilize a lead impedance sensor to measure a lead impedance between lead the 102 and the PG 100. In alternative embodiments, referring to FIG. 1, the sensor module 504 utilizes a force sensor to measure a force between the lead 102 and the PG 100. In embodiments, if more than one lead is connected to the PG 100, the sensor module 504 measures at least one characteristic associated with each lead connection to the PG 100.

In embodiments, the comparing module 506 receives measurements from the sensor module 504 and compares the measurements with a threshold. As an example, if the comparing module 506 receives one or more lead impedance measurements from the sensor module 504, the comparing module 506 compares the received lead impedance measurement(s) against a preprogrammed lead impedance threshold. As another example, if the comparing module 506 receives one or more force measurements from the sensing module 504, the comparing module 506 compares the received force measurement(s) against a predetermined force threshold.

In some embodiments, the timer module 508 receives commands from the comparing module 506 to start and stop a timer. As an example, when the comparing module 506 initially determines that a measured lead impedance exceeds a lead impedance threshold, the comparing module 506 sends a command to the timer module 508 to start a timer. When the comparing module 506 determines that the measured lead impedance falls below the lead impedance threshold, after the measured lead impedance exceeded the lead impedance threshold, the comparing module 506 sends a command to the timer module 508 to stop the timer. In another example, when the comparing module 506 determines that a measured force falls below a force threshold, the comparing module 506 sends a command to the timer module 508 to initiate the timer. If the comparing module 506 determines that the measured force is above the force threshold after previously falling below the force threshold, the comparing module 506 sends a command to the timer module 508 to stop the timer. In some embodiments, each instance the timer module 508 receives a command to stop the timer after previously receiving a command to initiate the timer, the timer module 508 resets the timer.

In some embodiments, the flag setting module 510 receives the command to set an MRI authorization flag and a lead detection flag. In embodiments, when the timer module 508 determines that the timer has exceeded a specified period of time (e.g., five seconds), the timer module 508 sends a command to the flag setting module 510 to set the MRI authorization flag to an unapproved state (e.g., 0). In embodiments, the lead detection module 502 sends a command to the flag setting module 510 upon detection of a lead being attached to the PG. In embodiments, upon setting the MRI authorization flag to an unapproved state, the lead detection flag is set low, the sensor module 504 discontinues performing measurements, and the lead detection module 502 starts the process for checking for a new lead connection.

In some embodiments, the system 500 includes an MRI authorization flag and a lead detection flag for each lead connection to the PG. In embodiments, for each lead connection detected by the lead detection module 502, a lead connection flag is set high and the sensor module 504 starts measuring the impedance/force for each detected lead connection. For each lead disconnect detected, the MRI authorization flag and lead detection flag for that disconnected lead is set high and low, respectively. Upon setting the lead detection flag low for a particular lead, the sensor module discontinues performing lead/force measurements for that lead, and the lead detection module 502 starts the process for detecting a new lead connection for that lead. Accordingly, the lead detection module 502 searches for new lead connections for leads where the lead detection flag is set low, and the sensor module 504 performs lead/force measurements for leads where the lead detection flag is set high.

In some embodiments, the communications module 512 outputs a signal upon receiving a command from the flag setting module 510 indicating that the MRI authorization flag has been set to an unapproved state. As an example, when the system 500 of FIG. 5 is located in the PG 100 of FIG. 1, the communications module 512 outputs the signal to the remote terminal 108 to indicate that the PG configuration of PG 100 may be MRI conditionally unsafe. In embodiments, the checking module 514 checks to see if the MRI authorization flag is in an unapproved state. As an example, the checking module 514 is initiated prior to conducting an MRI on a patient that has the PG 100.

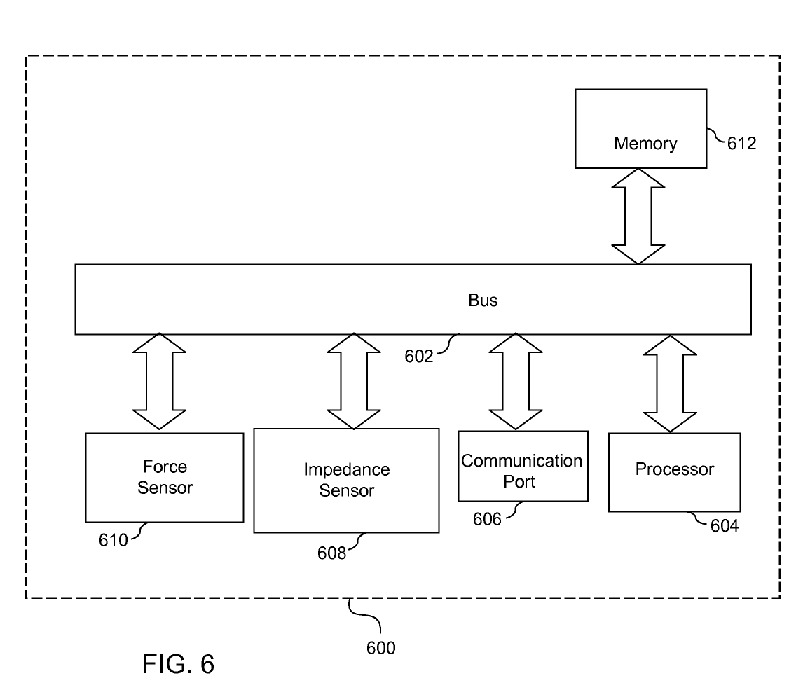

FIG. 6 is a schematic diagram of an example computing device 600 upon which embodiments of the present invention may be implemented. In embodiments, the computing device 600 implements each of the modules illustrated in FIG. 5.

According to the present example, the computing device 600 includes a bus 602, at least one processor 604, a communication port 606, an impedance sensor 608, a force sensor 610, and a memory 612. In embodiments, each of these components are interfaced with the bus 602 and configured to communicate with each other via the bus 602.

Processor(s) 604 can be any desired processor, such as, but not limited to Z80, ARM, ARC, or any hardware based micro coded sequencer. Communication port(s) 606 can be any desired port suitable for facilitating communication between the PG 100 and remote terminal 108 of FIG. 1. As an example, communication port 606 is a wireless (RF) transmitter or an acoustic transducer.

In embodiments, the processor 604 is configured to execute each of the example modules illustrated in FIG. 5. In embodiments, the processor 604 is configured to control the impedance sensor 608 to measure the lead impedance between a lead and a PG. In embodiments, the processor 604 is configured to control the force sensor 610 to measure the force between a lead and a PG.

Memory 612 may comprise a Random Access Memory (RAM) or any other suitable dynamic storage device(s). In some embodiments, the processor 604 utilizes the memory 612 to execute each of the modules illustrated in FIG. 5. Bus 602 communicatively couples processor(s) 604 with the other memory, storage and communication blocks.

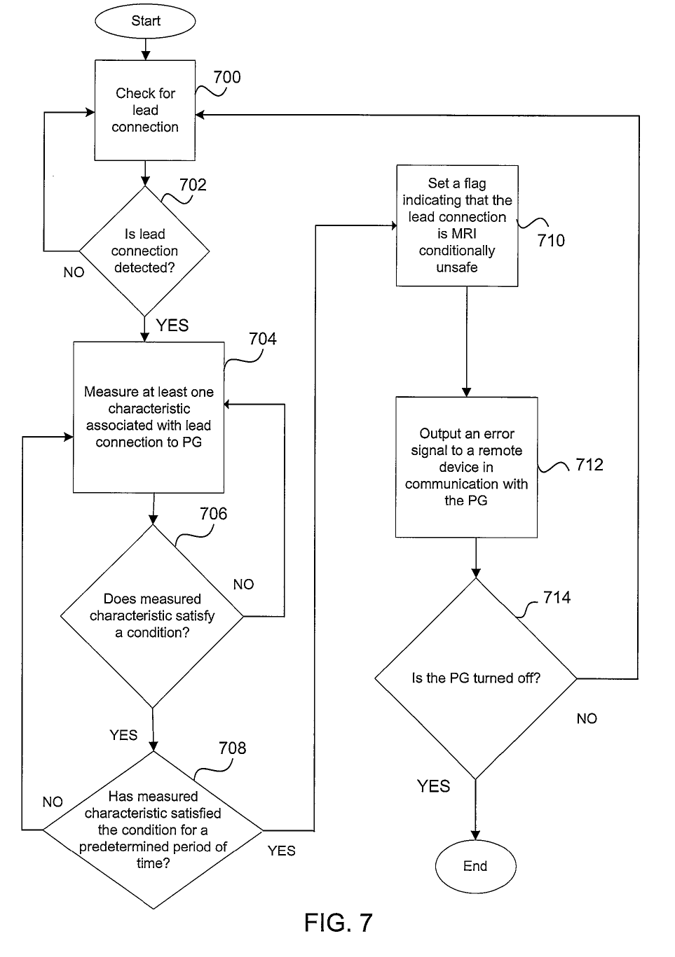

FIG. 7 is a flow chart illustrating an example method for detecting the disconnect of a lead from a PG. In embodiments, referring to FIG. 1, the method illustrated in FIG. 6 is implemented as a routine or algorithm on the PG 100 and/or the remote terminal 108. In embodiments, the method illustrated in FIG. 7 is implemented by the computing device 600 illustrated in FIG. 6.

The method may begin generally at block 700 by the PG checking for a lead connection to the PG (e.g. determining whether a lead is connected to the PG). In embodiments, for example, the lead detection module 502 performs impedance and/or force measurements, as described above, to detect whether a lead is connected to the PG. If the PG has not detected a lead connection 702, the PG continues checking for a lead connection 600 until the lead connection is detected.

Upon detecting the lead connection to the PG, the PG or remote terminal starts measuring at least one characteristic associated with the lead connection to the PG 704. In embodiments, the sensor module 504 utilizes a lead impedance sensor to measure the lead impedance between the lead and the pulse generator. In alternative embodiments, the sensor module 504 utilizes a force sensor to measure the force between the lead and the pulse generator.

Upon measuring the at least one characteristic associated with the lead connection to the PG, the PG or remote terminal determines whether the measured characteristic satisfies a condition 706. For example, when the measured characteristic is the lead impedance between the lead and the PG, a condition is satisfied when the measured lead impedance is above a lead impedance threshold. In other embodiments, when the measured characteristic is the force between the lead and the PG, a condition is satisfied when the measured force is below a force threshold. In embodiments, the comparing module 506 performs the comparisons. If the measured characteristic does not satisfy the condition, the PG or remote terminal continues measuring the at least one characteristic associated with the lead connection to the PG 704.

Upon determining if the measured characteristic satisfies the condition, the PG or remote terminal determines if the measured characteristic has satisfied the condition for a predetermined period of time. In embodiments, upon determination that a condition is satisfied, the timer module 508 starts a timer. A lead disconnect is detected upon the timer reaching the predetermined period of time. If the measured characteristic has not satisfied the condition for the predetermined period of time, the PG or remote terminal continues measuring the at least one characteristic associated with the lead connection to the PG 704.

Upon determining that the measured characteristic satisfied the condition for the predetermined period of time, the PG or remote terminal sets a flag to indicate that the lead wires or the PG may be MRI conditionally unsafe 710. In embodiments, the flag setting module 510 sets the MRI authorization flag to an unapproved state upon determining that a measured lead impedance between a lead and a PG has exceeded a lead impedance threshold for the predetermined period of time. In alternative embodiments, the flag setting module 510 of FIG. 5 sets the MRI authorization flag to an unapproved state upon determination that a measured force between a lead and a PG has fallen below a force threshold for the predetermined period of time.

Upon setting the flag, the PG outputs an error signal to a remote device in communication with the PG 712. As an example, referring to FIG. 1, when the MRI authorization flag is set to an unapproved state and the measurements are stopped by the PG 100, an error signal is outputted from the PG 100 to the remote terminal 108. In embodiments, the error signal indicates that the lead wires or the PG may be MRI conditionally unsafe.

If the PG is turned off at 714 after outputting the error signal to the remote device, the process illustrated in FIG. 7 ends. If the PG is not turned off 714 after outputting the error signal to the remove device, the PG returns to checking for a lead connection 700. Accordingly, as illustrated in FIG. 7, after the MRI authorization flag is set high, which occurs when a lead disconnect has been detected, the PG returns to checking for the next lead connection if the PG has not been turned off. Further, in embodiments, the process illustrated in FIG. 7 ends automatically at any point in the process when the PG is turned off.

In embodiments, when there is more than one lead connection to the PG, the process illustrated in FIG. 7 is performed in parallel for each lead connection. As an example, when the PG detects a first lead connection 702, the PG continues the execution of the process illustrated in FIG. 7 for the first lead connection by measuring at least one characteristic associated with the first lead connection 704. The PG will continue to check for the next lead connection in parallel with the execution of the process illustrated in FIG. 7 for the first lead connection. If the PG detects a second lead connection, the PG continues the execution of the process illustrated in FIG. 7 for the second lead connection in parallel with the execution of the process illustrated in FIG. 7 for the first lead connection.

Embodiments of the present invention include various steps, which are described herein. The steps may be performed by hardware components or may be embodied in machine-executed restrictions, which may be used to cause a general-purpose or special-purpose processor programmed with the instructions to perform the steps.

Various modifications and additions can be made to the example embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

What is claimed is:

1. A method for checking the connection of a lead to a implantable medical device implanted within a patient's body, the method comprising:
measuring at least one characteristic associated with the lead connection to the implantable medical device prior to an MRI scan;
comparing the at least one measured characteristic with a threshold parameter programmed within the implantable medical device; and
setting a flag in the implantable medical device upon the at least one measured characteristic satisfying at least one condition associated with the threshold parameter for a predetermined period of time, the flag indicating a disconnection of the lead from the implantable medical device.

2. The method of claim 1, further comprising:
detecting the lead connection to the implantable medical device.

3. The method of claim 1, further comprising:
outputting an error signal to a remote device in communication with the implantable medical device, the error signal indicating the disconnection of the lead with the implantable medical device.

4. The method according to claim 1, wherein
said measuring the at least one characteristic further includes measuring a lead impedance parameter of the lead connection to the implantable medical device;
said threshold parameter includes a predetermined lead impedance parameter; and
said at least one condition includes the measured lead impedance parameter exceeding the predetermined lead impedance parameter.

5. The method according to claim 1, wherein
said measuring the at least one characteristic further includes measuring a force parameter between the lead connection and the implantable medical device;
said threshold parameter includes a predetermined force parameter; and
said at least one condition includes the measured force parameter decreasing below the predetermined force parameter.

6. The method according to claim 2, further comprising:
initiating said measuring the at least one characteristic upon detection of the lead connection to the implantable medical device;
discontinuing said measuring the at least one characteristic upon setting said flag; and
initiating detecting the lead connection to the implantable medical device upon discontinuing said measuring the at least one characteristic.

7. The method according to claim 6, wherein said measuring is uninterrupted between said initiating said measuring the at least one characteristic and said discontinuing said measuring the at least one characteristic.

8. The method according to claim 1, further comprising:
checking the flag prior to conducting said MRI on the lead connection; and
examining the lead connection upon determination that the flag is set.

9. The method of claim 1, implemented by a computer readable medium including executable computer instructions.

10. A system for checking the connection of a lead to a implantable medical device implanted within a patient's body, the system comprising:
a sensor module configured to measure at least one characteristic associated with the lead connection to the implantable medical device;
a comparing module configured to compare the at least one measured characteristic with a threshold; and
a flag setting module configured to set a flag upon the at least one measured characteristic satisfying at least one condition associated with said threshold for a predetermined period of time, the flag indicating a disconnection of the lead from the implantable medical device.

11. The system according to claim 10, further comprising:
a lead detection module configured to detect the lead connection to the implantable medical device.

12. The system according to claim 10, further comprising:
a communications module configured to output an error signal to a remote device in communication with the implantable medical device, the error signal indicating the disconnection of the lead with the implantable medical device.

13. The system according to claim 10, wherein said sensor module and said comparing module are incorporated in the implantable medical device.

14. The system according to claim 10, wherein
said sensor module is further configured to measure the lead impedance parameter of the lead connection to the implantable medical device,
said threshold includes a predetermined lead impedance parameter, and
said at least one condition includes said measured lead impedance parameter exceeding said predetermined lead impedance parameter.

15. The system according to claim 10, wherein
said measuring module is further configured to measure a force parameter between the lead connection and the implantable medical device;
said threshold includes a predetermined force parameter, and
said at least one condition includes said measured force parameter decreasing below said predetermined force parameter.

16. The system according to claim 11, wherein
said measuring module is further configured to start measuring the at least one characteristic upon said lead detection module detecting the lead connection to the implantable medical device;
said measuring module is further configured to discontinue measuring the at least one characteristic upon said flag setting module setting said flag; and
said lead detection module is further configured to start detecting the lead connection to the implantable medical device upon discontinuing said measuring module from measuring the at least one characteristic.

17. The system according to claim 16, wherein said measuring module is uninterrupted between starting said measuring module to measure the at least one characteristic and discontinuing said measuring from measuring the at least one characteristic.

18. The system according to claim 10, further comprising:
a checking module configure to check said flag prior to conducting an MRI on said lead connection.

19. An implantable medical device implanted within a patient's body, the implantable medical device comprising:
a sensor module configured to measure at least one characteristic associated with the connection of a lead to the implantable medical device;
a comparing module configured to compare the at least one measured characteristic with a threshold; and
a flag setting module configured to set a flag upon the at least one measured characteristic satisfying at least one condition associated with said threshold for a predetermined period of time, the flag indicating a disconnection of the lead from the implantable medical device.

20. The implantable medical device according to claim 19, further comprising:
a lead detection module configured to detect the lead connection to the implantable medical device.

21. The implantable medical device according to claim 19, further comprising:
a communications module configured to output an error signal to a remote device in communication with the implantable medical device, the error signal indicating the disconnection of the lead with implantable medical device.

22. The implantable medical device according to claim 19, wherein
said sensor module is further configured to measure a lead impedance parameter of the lead connection to the implantable medical device,
said threshold includes a predetermined lead impedance parameter, and
said at least one condition includes said measured lead impedance parameter exceeding said predetermined lead impedance parameter.

23. The implantable medical device according to claim 19, wherein
said measuring module is further configured to measure a force parameter between the lead connection and the implantable medical device;
said threshold includes a predetermined force parameter, and
said at least one condition includes said measured force parameter decreasing below said predetermined force parameter.

24. The implantable medical device according to claim 20, wherein
said measuring module is further configured to start measuring the at least one characteristic upon said lead detection module detecting the lead connection to the implantable medical device;
said measuring module is further configured to discontinue measuring the at least one characteristic upon said flag setting module setting said flag; and
said lead detection module is further configured to start detecting the lead connection to the implantable medical device upon discontinuing said measuring module from measuring the at least one characteristic.

25. The implantable medical device according to claim 24, wherein said measuring module is uninterrupted between starting said measuring module to measure the at least one characteristic and discontinuing said measuring from measuring the at least one characteristic.
